(19) United States
(12) Patent Application Publication
(10) Pub. No.: US 2011/0208094 A1
(43) Pub. Date: Aug. 25, 2011

(54) ULTRASOUND NEUROMODULATION OF THE RETICULAR ACTIVATING SYSTEM

(76) Inventor: David J. Mishelevich, Playa del Rey, CA (US)

(21) Appl. No.: 13/031,192

(22) Filed: Feb. 19, 2011

Related U.S. Application Data
(60) Provisional application No. 61/306,531, filed on Feb. 21, 2010.

(51) Int. Cl. A61N 7/00

(52) U.S. Cl. 601/2

(57) ABSTRACT

Disclosed are methods and systems and methods for neuromodulation of the Reticular Activating System using ultrasound to produce acute effects or Long-Term Potentiation (LTP) or Long-Term Depression (LTD). Included is control of direction of the energy emission, intensity, pulse duration, frequency, and phase/intensity relationships to targeting and accomplishing up-regulation and/or down-regulation. The invention can be applied for a variety of clinical purposes such as reversibly putting a patient to sleep or waking them up (for example, for the purpose of anesthesia) or reversibly putting a patient into a coma (for example for the purpose of protecting or rehabilitating the brain of the patient after a stroke or head injury).

### CROSS REFERENCE TO RELATED APPLICATIONS

This patent application claims priority to Provisional Patent Application No. 61/306531, filed Feb. 21, 2010, entitled “ULTRASOUND NEUROMODULATION OF THE RETICULAR ACTIVATING SYSTEM.” The disclosures of this patent application are herein incorporated by reference in their entirety.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation including one or more ultrasound sources for neuromodulation of target deep brain regions to up-regulate or down-regulate neural activity.

### BACKGROUND OF THE INVENTION

It has been demonstrated that focused ultrasound directed at neural structures can stimulate those structures. If neural activity is increased or excited, the neural structure is said to be up-regulated; if neural activated is decreased or inhibited, the neural structure is said to be down-regulated. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22 (2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3 (10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 300 Hz.) are inhibitory. High frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

An alternative approach is described by Deisseroth and Schneider (U.S. patent application Ser. No. 12/263,026 published as US 2009/0112133 A1, Apr. 30, 2009) in which modification of neural transmission patterns between neural structures and/or regions is described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24 (2):275-83 and Clement G T, Hynynen K (2002) A non-invasive method for focusing ultrasound through the human skull. Phys Med Biol 47: 1219-1236.). Ultrasound can be focused to 0.5 to 2 mm as TMS to 1 cm at best.

### SUMMARY OF THE INVENTION

It is the purpose of this invention to provide methods and systems for non-invasive neuromodulation of the Reticular Activating System using ultrasound to produce acute effects or Long-Term Potentiation (LTP) or Long-Term Depression (LTD). Included is control of direction of the energy emission, intensity, frequency, and phase/intensity relationships to targeting and accomplishing up-regulation and/or down-regulation. Use of ancillary monitoring or imaging to provide feedback is optional. In embodiments were concurrent imaging is to be done, the device of the invention is to be constructed of non-ferrous material.

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target. For example, some targets, like the Reticular Activating System, are elongated and will be more effectively served with an elongated ultrasound field at the target.

### BRIEF DESCRIPTION OF THE DRAWINGS

FIG. 1 shows saggital view of brain highlighting the Reticular Activating System including ultrasound transducer positioning.

FIG. 2 illustrates two alternative ultrasound transducer positions for targeting the Reticular Activating System.

FIG. 3 shows side and top views of pattern generated by the ultrasound transducer.

FIG. 4 shows a block diagram of the control circuit.

### DETAILED DESCRIPTION OF THE INVENTION

It is the purpose of this invention to provide methods and systems and methods for neuromodulation of the Reticular Activating System using ultrasound to produce acute effects or Long-Term Potentiation (LTP) or Long-Term Depression (LTD). Included is control of direction of the energy emission, intensity, frequency, and phase/intensity relationships to targeting and accomplishing up-regulation and/or down-regulation.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz with power generally applied less than 60 mW/cm² but also at higher target- or patient-specific levels at which no tissue damage is caused. The acoustic frequency is gated at the lower rate to impact the neuronal structures as desired (e.g., say 300 Hz for inhibition (down-regulation) or 1 kHz for excitation (up-regulation). Ultrasound therapy can be combined with therapy using other devices such as medications, electrical stimulation, application of optogenetics, local anesthetic blocks, surgical interventions, radiosurgery, cryotherapy, Radio-Frequency (RF) therapy and Transcranial Magnetic Stimulation (TMS).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well. Ultrasound conduction medium will be required to fill the space. An example of suitable ultrasound conduction medium is Dermasol from California Medical Innovations.

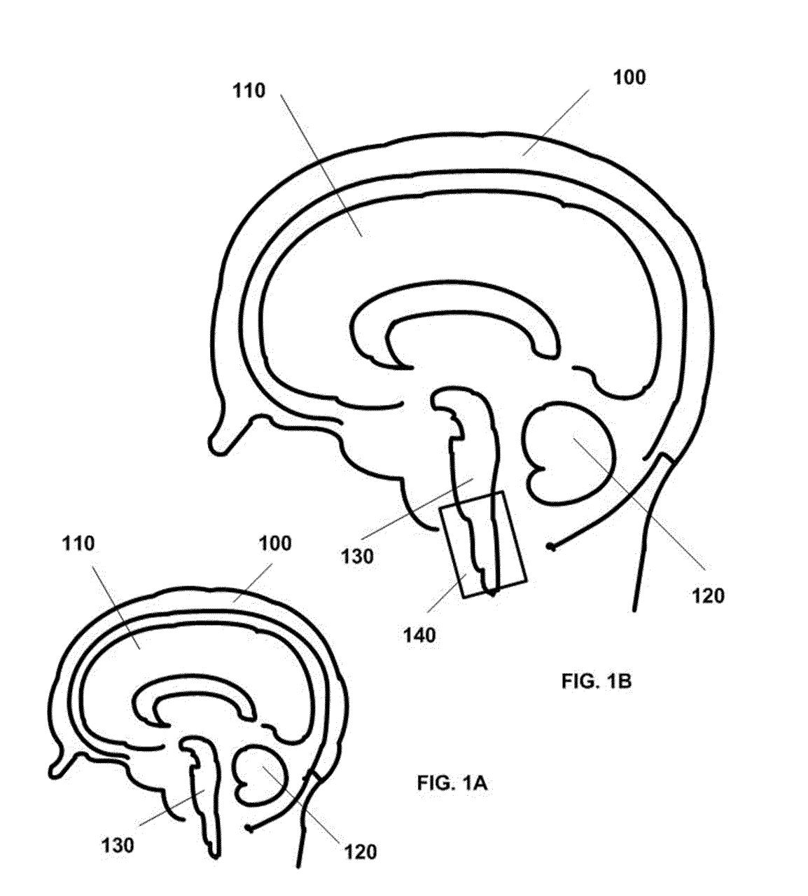

FIG. 1A shows sagittal view of brain highlighting the Reticular Activating System 130 including skull 100 with cerebrum 110 along with cerebellum 120. FIG. 1B again shows the Reticular Activating System 130 including skull 100 with cerebrum 110 along with cerebellum 120, but this time with ultrasound transducer 140 approximately aligned along the axis of the Reticular Activating System and placed against the neck. The ultrasound transducer 140 does not cover the entire length of the Reticular Activating System (RAS) first because the upper part of the is not physically accessible (although the top of the outline 130 is the midbrain which is outside the RAS) and second because the ultrasound field can be steered to a point above the top of the ultrasound transducer 140. In another embodiment, the ultrasound transducer is perturbed laterally, up and down, and/or in and out causing enhanced change in the target neural tissue.

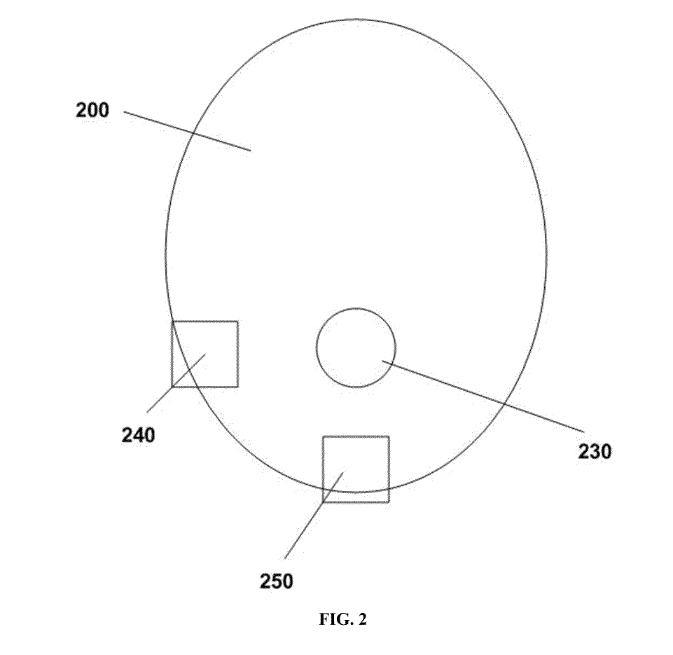

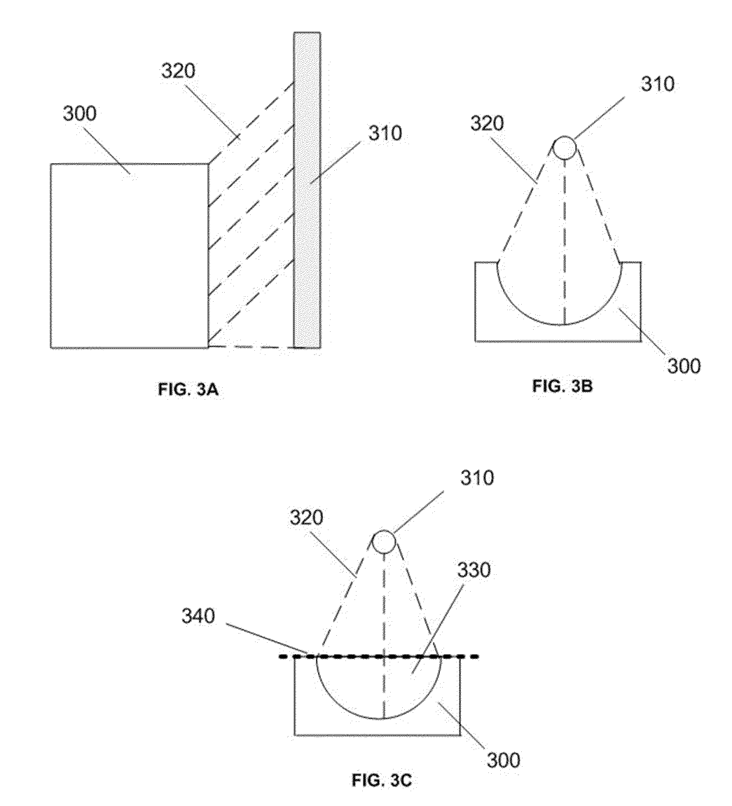

FIG. 2 shows the top view of patient head 200 showing two embodiments of ultrasound transducer placements with respect to Reticular Activating System 230, the first in which the ultrasound transducer is placed laterally 240 to RAS 230 and against the patient's neck and the second in which the ultrasound transducer 250 is placed posterior to RAS 230 against the patient's neck. Note that the placement of lateral ultrasound transducer 230 can be to the right of RAS 230 or to its left. For the ultrasound to be effectively transmitted through the tissues to the RAS target, coupling must be put into place. Ultrasound transmission medium (e.g., Dermasol from California Medical Innovations or silicone oil in a containment pouch) (not shown) is interposed with one mechanical interface to the ultrasound transducer, either 240 or 250 completed by a layer of ultrasound transmission gel (not shown). The depth of the point where the ultrasound is focused depends on the shape of the transducer and setting of the phase and amplitude relationships of the elements of the ultrasound transducer array discussed in relation to FIG. 3. In other embodiments, ultrasound transducers may be place on both sides of the patient's neck. In a further embodiment, multiple ultrasound transducers may be used either in the vertical direction, horizontal direction, or both.

FIG. 3A shows a lateral view of ultrasound transducer 300 with its ultrasound pattern 320 aimed at the Reticular Activating System (RAS) target 310. Ultrasound-transducer field 320 is steered upwards by controlling the phase/intensity relationships of the array elements of ultrasound transducer 300 so it can hit target 310 at a point that is superior to the top of ultrasound transducer 300. Transducer array assemblies of this type may be supplied to custom specifications by Imasonic in France (e.g., large 2D High Intensity Focused Ultrasound (HIFU) hemispheric array transducer) (Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Blatek and Keramos-Etalon in the U.S. are other custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations. FIG. 3B shows a plan view of the configuration with ultrasound transducer 300, RAS target 310, and ultrasound pattern 320. FIG. 3C shows the configuration where added to ultrasound transducer 300, RAS target 310, and ultrasound pattern 320 are ultrasound conduction medium 330 contained within the transducer, and ultrasound conducting gel layer 340 which is pressed against the skin of the patient. In another embodiment, a plurality of ultrasonic transducers is aimed at the Reticular Activating System.

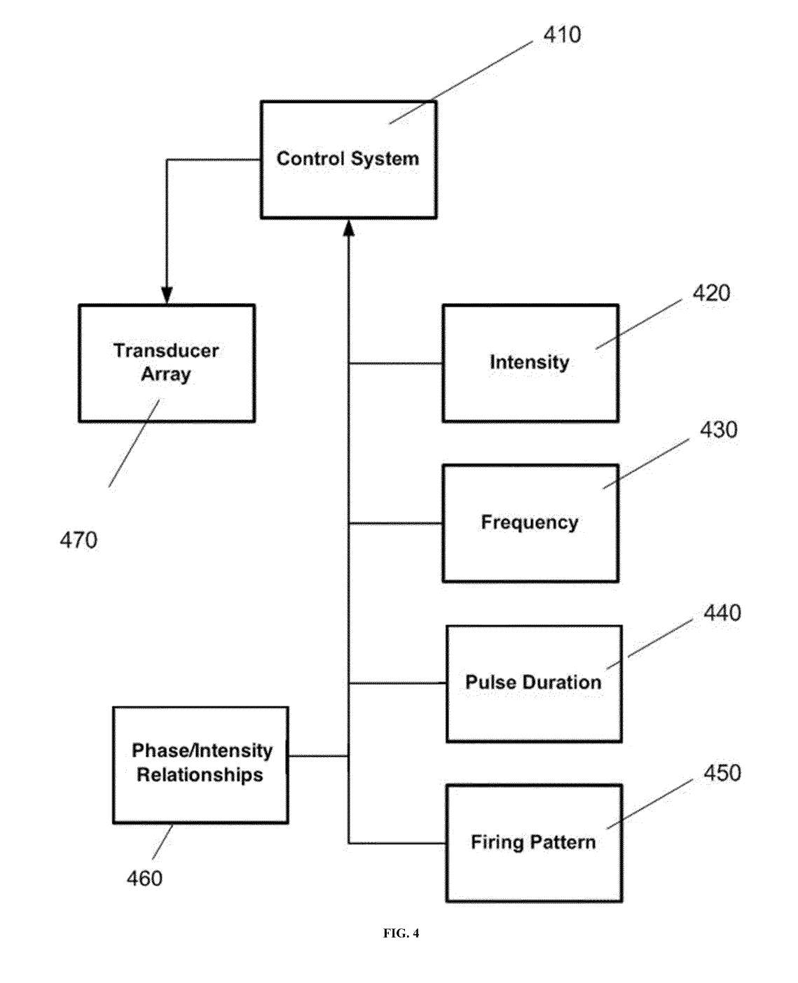

FIG. 4 illustrates the control circuit. Control System 410 receives its input from Intensity setting 420, Frequency setting 430 for up regulation or down regulation, Pulse-Duration setting 440, Firing-Pattern setting 450, and Phase/Intensity Relationships 460 for beam steering and focusing on neural targets. Control System 410 then provides output to drive Transducer Array 470 and thus deliver the neuromodulation.

In still another embodiment movement of the transducer and/or controlling stimulation parameters and seeing the physiological response of the patient is used to correctly locate the Reticular Activating System.

In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback. In still another embodiment, RF emitters are used in place or ultrasound transducers.

The invention can be applied for a variety of clinical purposes such as reversibly putting a patient to sleep or waking them up (for example, for the purpose of anesthesia) or reversibly putting a patient into a coma (for example for the purpose of protecting or rehabilitating the brain of the patient after a stroke or head injury). Effects can be either acute or durable effect through Long-Term Potentiation (LTP) and/or Long-Term Depression (LTD). Since the effect is reversible putting the patient in even a vegetative state is safe if handled correctly. The application of LTP or LTD provides a mechanism for adjusting the bias of patient activity up or down. Appropriate radial (in-out) positions can be determined through patient-specific imaging (e.g., PET or fMRI) or set based on measurements to the mid-line. The positions can set manually or via a motor (not shown). The invention allows stimulation adjustments in variables such as, but not limited to, intensity, firing pattern, pulse duration, frequency, phase/intensity relationships, dynamic sweeps, and position.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of deep-brain ultrasound stimulation, the method comprising:
aiming one or an plurality of ultrasound transducer at the Reticular Activating System,
applying pulsed power to the one or a plurality of ultrasound transducers via a control circuit
whereby the Reticular Activating System is neuromodulated.

2. The method of claim 1, further comprising locating the Reticular Activating System.

3. The method of claim 1, wherein the plurality of control elements is selected from the group consisting of intensity, frequency, pulse duration, firing pattern, and phase/intensity relationships.

4. The method of claim 1, further comprising aiming an ultrasound transducer neuromodulating the Reticular Activating System in a manner selected from the group of up-regulation, down-regulation.

5. The method of claim 1, wherein the acoustic ultrasound frequency is in the range of 0.3 MHz to 0.8 MHz.

6. The method of claim 1, where in the power applied is less than 60 mW/cm².

7. The method of claim 1, wherein the power applied is greater than 60 mW/cm² but less than that causing tissue damage.

8. The method of claim 1, wherein a stimulation frequency for of 300 Hz or lower is applied for inhibition of neural activity.

9. The method of claim 1, wherein the stimulation frequency for excitation is in the range of 500 Hz to 5 MHz.

10. The method of claim 1, wherein the focus area of the pulsed ultrasound is 0.5 to 50 mm in diameter.

11. The method of claim 1, wherein the focus area of the pulsed ultrasound is 50 to 150 mm in diameter.

12. The method of claim 1, wherein the step of aiming comprises aiming a plurality of ultrasonic transducers at the Reticular Activating System.

13. The method of claim 1, wherein the number of ultrasound transducers is between 1 and 5.

14. The method of claim 1, wherein the clinical function is selected from the group consisting of: reversibly putting a patient to sleep or waking them up and reversibly putting a patient into a coma.

15. The method of claim 1, wherein the clinical purpose is selected from the group consisting of: anesthesia, protecting or rehabilitating the brain after a stroke, and protecting or rehabilitating the brain after head trauma.

16. The method of claim 1, wherein mechanical perturbations are applied radially or axially to move the ultrasound transducers.

17. The method of claim 1, wherein the neuromodulation results in a durable effect selected from the group consisting of Long-Term Potentiation and Long-Term Depression.

18. The method of claim 1, wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, patient.

19. The method of claim 1, wherein RF emitters are used in place or ultrasound transducers.

20. The method of claim 1, wherein ultrasound therapy is combined with one or more therapies selected from the group consisting of medications, electrical stimulation, application of optogenetics, local anesthetic blocks, surgical interventions, radiosurgery, cryotherapy, Radio-Frequency (RF) therapy and Transcranial Magnetic Stimulation (TMS).
